(19) United States
(12) Patent Application Publication
(10) Pub. No.: US 2024/0149037 A1
(43) Pub. Date: May 9, 2024

(54) Stabilization Device with Injection Position Rotation for Self-Cannulation

(71) Applicant: Bard Peripheral Vascular, Inc., Franklin Lakes, NJ (US)

(72) Inventors: Adam T. Martin, Holly Springs, NC (US); Nicholas J. Jardine, Holly Springs, NC (US)

(21) Appl. No.: 18/280,182

(22) PCT Filed: Mar. 9, 2022
(86) PCT No.: PCT/US22/19602

§ 371 Date: Sep. 1, 2023

Related U.S. Application Data
(60) Provisional application No. 63/159,189, filed on Mar. 10, 2021.

(51) Int. Cl. A61M 39/02; A61M 5/158

(52) U.S. Cl. CPC A61M 39/0208; A61M 5/158; A61M 2005/1585; A61M 2005/1586; A61M 2039/0238; A61M 2209/04

(57) ABSTRACT

Embodiments disclosed herein are directed to a stabilization device (100) configured to engage a subcutaneous medical device, or port (40). The device can include a guide cylinder (120) that aligns with a receiving cup (46) of the port when the port is engaged with a bottom surface (116) of the stabilization device. The guide cylinder can be rotatable between a first position and a second position. The guide cylinder can further include a needle channel (122) extending therethrough. An axis of the needle channel can be offset from the central axis of the guide cylinder. As such rotating the guide cylinder between the first position and the second position can align the needle channel with a different insertion site. The needle channel can align with the receiving cup of the port in both the first position and the second position. Accessing the receiving cup through different insertion sites can mitigate the formation of scar tissue.

### PRIORITY

This application claims the benefit of priority to U.S. Provisional Application No. 63/159,189, filed Mar. 10, 2021, which is incorporated by reference in its entirety into this application.

### SUMMARY

Briefly summarized, embodiments disclosed herein are directed to a stabilization device with injection position rotation for self-cannulation for cannulating a subcutaneous access device.

Dialysis patients typically travel to dialysis treatment centers to receive their treatment. This can occur multiple times per week (e.g. between three to seven times per week) and typically require the patient to remain on site for several hours at a time. Some patients however have the option to perform the dialysis therapy from the comfort of their home. A major concern with accessing a subcutaneous dialysis port with a needle or cannula device, is that over multiple accesses in the same location, the patient can develop scar tissue, or degrade the skin tissues around the injection site creating a “buttonhole.” The degraded skin can significantly increase the risk of infection at the insertion site. To mitigate the creation of scar tissue, or “buttonholes,” a clinician can rotate the position of the insertion site. In a home setting, rotating the insertion site can be challenging, especially if the patient is performing the access insertion themselves.

Embodiments disclosed herein are directed to a device configured to guide a user to rotate through different insertion sites to mitigate the creation of scar tissue or “buttonholes.”

Disclosed herein is a stabilization device for a subcutaneous port including, a body defining a port recess disposed in a bottom surface thereof and configured to receive a portion of the subcutaneous port therein in a first orientation, and a guide cylinder including a needle channel aligned with a receiving cup of the port when the port is engaged with the body in the first orientation, the guide cylinder extending from an outer surface of the body to the port recess and rotationally coupled to the body between a first position and a second position, the needle channel aligned with a first insertion site on the skin surface in the first position and a second insertion site on the skin surface in the second position.

In some embodiments, a central axis of the needle channel is offset from a central axis of the guide cylinder.

In some embodiments, the stabilization device further includes a dial, rotationally coupled with the body and configured to rotate the guide cylinder between the first position and the second position.

In some embodiments, an inner surface profile of the port recess is configured to mirror an outer profile of the port to align the guide cylinder with the receiving cup of the port in the first orientation and to prevent the port from engaging the port recess in a second orientation.

In some embodiments, an arc distance between the first position and the second position is between 1° and 180°.

In some embodiments, an arc distance between the first position and the second position is a non-factorable number of degrees of 360°.

In some embodiments, an arc distance between the first position and the second position is a prime number.

In some embodiments, the guide cylinder is configured to automatically rotate from the first position to the second position when a needle is removed from the needle channel.

In some embodiments, the stabilization device further includes a ratchet mechanism configured to prevent the guide cylinder from rotating from the second position to the first position.

In some embodiments, the needle channel moves in a circular path between the first position and the second position.

In some embodiments, the needle channel moves in a non-circular path between the first position and the second position.

Also disclosed is a method of accessing a subcutaneous port including, engaging a stabilization device with the subcutaneous port to axially align a guide cylinder of the stabilization device with a receiving cup of the port, inserting a needle through a needle channel of the guide cylinder to access the receiving cup of the subcutaneous port at a first insertion site, removing the needle from the stabilization device, and rotating the guide cylinder of the stabilization device from a first position to a second position to align the needle channel with a second insertion site, different from the first access site.

In some embodiments, a central axis of the needle channel is offset from a central axis of the guide cylinder.

In some embodiments, the method further includes rotating a dial from a first position to a second position to rotate the guide cylinder from the first position to the second position.

In some embodiments, an inner surface profile of a port recess of the stabilization device is configured to mirror an outer profile of the subcutaneous port to align the guide cylinder with the receiving cup of the subcutaneous port in a first orientation and to prevent the port recess from engaging the port in a second orientation, different from the first orientation.

In some embodiments, an arc distance between the first position and the second position of the guide cylinder is between 1° and 180°.

In some embodiments, an arc distance between the first position and the second position of the guide cylinder is a non-factorable number of degrees of 360°.

In some embodiments, an arc distance between the first position and the second position of the guide cylinder is a prime number.

In some embodiments, removing the needle from the needle channel causes the guide cylinder to automatically rotate from the first position to the second position.

In some embodiments, the stabilization device includes a ratchet mechanism configured to prevent the guide cylinder from rotating from the second position to the first position.

In some embodiments, the method further includes rotating the needle channel in a circular path between the first position and the second position.

In some embodiments, the stabilization device further includes rotating the needle channel in a non-circular path between the first position and the second position.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a perspective view of a stabilization device disposed on a skin surface, in accordance with embodiments disclosed herein.

FIG. 2 shows an exploded view of a stabilization device and port assembly, in accordance with embodiments disclosed herein.

FIGS. 3A-3B show a front, perspective view of a stabilization device in various rotational positions, in accordance with embodiments disclosed herein.

FIG. 4 shows a cross section view of the stabilization device engaged with a subcutaneous port, in accordance with embodiments disclosed herein.

FIGS. 5A-5B show a schematic view of various rotational positions of a guide cylinder and needle channel, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a needle disclosed herein includes a portion of the needle intended to be further from a patient, when the needle is used on the patient. Likewise, a “proximal length” of, for example, the needle includes a length of the needle intended to be further from the patient when the needle is used on the patient. A “proximal end” of, for example, the needle includes an end of the needle intended to be further from the patient when the needle is used on the patient. The proximal portion, the proximal end portion, or the proximal length of the needle can include the proximal end of the needle; however, the proximal portion, the proximal end portion, or the proximal length of the needle need not include the proximal end of the needle. That is, unless context suggests otherwise, the proximal portion, the proximal end portion, or the proximal length of the needle is not a terminal portion or terminal length of the needle.

With respect to “distal,” a “distal portion” or a “distal end portion” of, for example, a needle disclosed herein includes a portion of the needle intended to be near or in a patient when the needle is used on the patient. Likewise, a “distal length” of, for example, the needle includes a length of the needle intended to be near or in the patient when the needle is used on the patient. A “distal end” of, for example, the needle includes an end of the needle intended to be near or in the patient when the needle is used on the patient. The distal portion, the distal end portion, or the distal length of the needle can include the distal end of the needle; however, the distal portion, the distal end portion, or the distal length of the needle need not include the distal end of the needle. That is, unless context suggests otherwise, the distal portion, the distal end portion, or the distal length of the needle is not a terminal portion or terminal length of the needle.

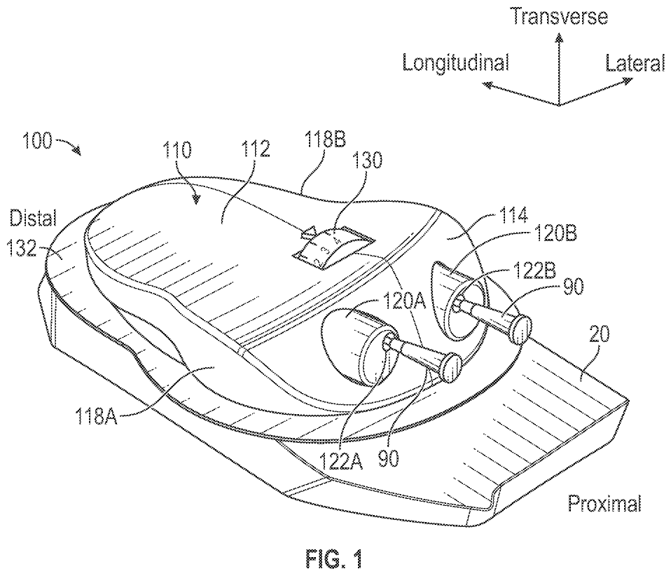

To assist in the description of embodiments described herein, as shown in FIG. 1, a longitudinal axis extends substantially parallel to an axial length of the needle channel 122. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

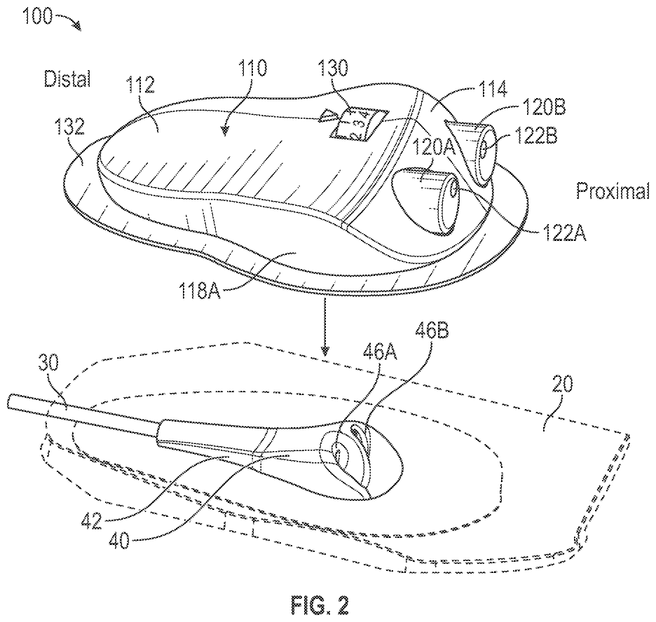

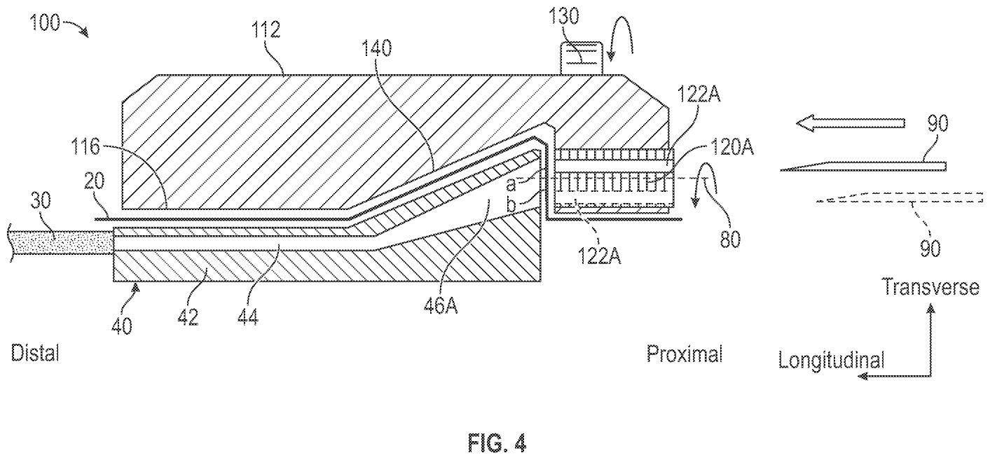

FIGS. 1-4 show an embodiment of a stabilization device (“device”) 100 configured to engage with a subcutaneous access device, or “port” 40 and facilitate accessing the port 40 with a needle 90 through various insertion sites. FIGS. 2 and 4 show an exemplary subcutaneous access device, such as a port 40. However, it will be appreciated that other subcutaneous, needle-accessible, medical devices are also contemplated to fall within the scope of the invention. As used herein, the port 40 can be a subcutaneous vascular access port, or similar subcutaneous medical device, accessible by a needle 90. The port 40 can be in fluid communication with a catheter 30. A distal tip of the catheter 30 can be disposed within a vasculature of a patient and provide fluid communication therewith.

As shown in FIGS. 2 and 4, the port 40 can generally include a port body 42 defining a conduit 44 that is in fluid communication with a lumen of a catheter 30 at a distal end thereof. A proximal end of the conduit 40 can define a funnel shaped, receiving cup 46 defining a tapered profile and configured to direct a needle 90 inserted therein, into the conduit 44. In an embodiment, the port 40 can include a needle penetrable septum, a needle penetrable valve, check valve, flap valve, duck-billed valve, slit valve, or the like, configured to control a fluid flow through the port 40. Optionally, the port 40 can include a reservoir in fluid communication with the conduit 44. The port 40 can define a transverse height and can define an outer profile configured to be palpated by a user to locate the port 40. In an embodiment, the port 40 can define a distinctive outer profile to facilitate orientation of the port 40 and location of the receiving cup 46 for a user to insert the needle 90 therein.

As shown in FIG. 1, the stabilization device 100 can include a body 110 defining at least a top surface 112, a proximal insertion surface 114 extending at an angle relative to the top surface 112, a bottom surface 116, a first side surface 118A, and a second side surface 118B disposed opposite the first side surface 118A. Optionally, the first side surface 118A or the second side surface 118B can include a profile and/or include a gripping feature configured to facilitate grasping the device 100 by a user. As shown, exemplary profiles can include indentations or concave sections configured to facilitate grasping of the body 210 by a user. Exemplary gripping features can include ridges, grooves, or different materials that have a relatively higher frictional co-efficient that the material of the body 110, e.g. silicone rubber, or the like.

In an embodiment, as shown in FIG. 4, the body 110 can further include a port recess 140 disposed in a bottom surface 116 and configured to receive a portion of a subcutaneous port 40 therein. The port recess 140 can define a geometry or profile configured to align the port 40 with the device 100 in a “correct” or first orientation, such that a guide cylinder 120 of the device 100 is aligned with a receiving cup 46 of the port 40. As such, at least a portion of the port 40, disposed subcutaneously, can engage the port recess 140 in the first orientation but can prevent the port 40 from engaging the port recess 140 in a second orientation, different from the first orientation. In an embodiment, the port recess 140 of the device 100 can define a similar concave inner profile, mirroring the convex outer profile of the port body 42. In an embodiment, the outer profile of the port 40 can define a distinct shape that can only be received within the port recess 140 when the port 40 is correctly aligned with the device 100 in the first orientation, but can prevent being received within a second orientation, different from the first orientation. In an embodiment, one or more protrusion of the port 40 can be configured to be received by one or more recess within the port recess 140 to align the port 40 with the device 100 in a “lock-and-key” style engagement.

In an embodiment, the device 100 can further include one or more guide cylinders 120, defining a substantially cylindrical shape with a circular cross-section. The guide cylinder 120 can extend through a portion of the body 110, extending from the proximal insertion surface 114 to the port recess 140. The guide cylinder 120 can be rotationally engaged with device body 110. An axis 80 of the guide cylinder 120 can be aligned with the receiving cup 46 when the port 40 is engaged with the device 100 in the first orientation.

In an embodiment, the device 100 can include a first guide cylinder 120A and a second guide cylinder 120B. Each guide cylinder 120 can align with a receiving cup 46 of the port 40. For example, the first guide cylinder 120A can align with a first receiving cup 46A that is in fluid communication with a first conduit of the port 40 and a first lumen of a catheter 30. The second guide cylinder 120B can align with a second receiving cup 46B that is in fluid communication with a second conduit of the port 40 and a second lumen of a catheter 30. As shown, the device 100 engages with a dual lumen port/catheter assembly, however it will be appreciated that this is exemplary and not intended to be limiting, and single lumen port/catheter assemblies or multi-lumen port/catheter assemblies are also contemplated to fall within the scope of the invention.

In an embodiment, the guide cylinder 120 can further include a needle channel 122 extending therethrough and configured to receive a needle 90 slidably engaged therewith. For example, a first guide cylinder 120A can include a first needle channel 122A and a second guide cylinder 120B can include a second needle channel 122B. The needle channel 122 can extend axially through the guide cylinder 120 from a proximal end to a distal end. The axis of the needle channel 122 can be offset from a central axis 80 of the guide cylinder 120. As such, rotating the guide cylinder 120 about the central axis 80 can align the axis of the needle channel 122 with a different portion of skin, as described in more detail herein.

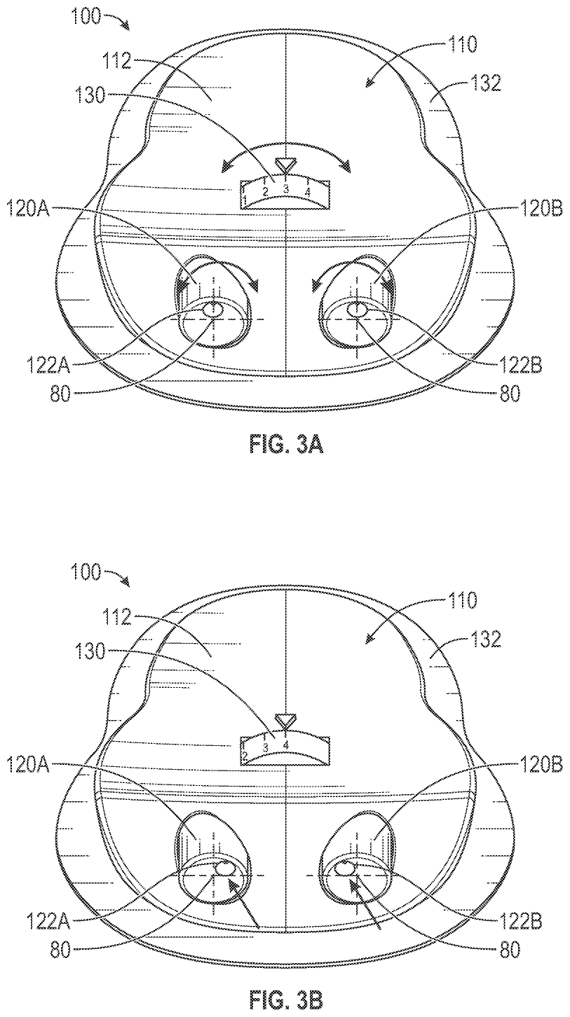

In an embodiment, the device 100 can further include a dial 130 rotatably engaged with a surface of the body 110, e.g. a top surface 112, and rotatable between two or more positions. In an embodiment, as shown in FIGS. 3A-3B, the dial 130 can be configured to rotate the guide cylinder(s) 120 about the central axis 80 of the guide cylinder 120 between two or more positions. In an embodiment, the dial 130 can be coupled with one or more guide cylinders 120 by way of one or more gear mechanisms, lever systems, biasing members, ratchets, pawls, or the like. As such, by rotating the dial 130 from a first position, e.g. FIG. 3A, to a second position, e.g. FIG. 3B, the dial 130 can rotate the guide cylinders 120 about the central axis 80 from a first position, e.g. FIG. 3A, to a second position, e.g. FIG. 3B.

As noted, the axis of the needle channel 122 can be offset from a central axis 80 of the guide cylinder 120. As such, rotating the guide cylinder 120 from the first position to the second position can align the needle channel 122 with a different portion of skin. As shown in FIG. 4, with the first guide cylinder 120A in the first position, the first needle channel 122A, extending therethrough, aligns with the first receiving cup 46A at a first insertion site “a.” Inserting a needle 90 through the first needle channel 122A in the first position can pierce the skin surface 20 at the first insertion site “a” and access the first receiving cup 46A. Rotating the first guide cylinder 120A about the longitudinal axis, from the first position to a second position aligns the first needle channel 122A with a second insertion site “b”. Inserting the needle 90 through the first needle channel 122A in the second position, the needle 90 can access the first receiving cup 46A by penetrating the skin at the second insertion site “b.” Advantageously, the stabilization device 100 can align the needle 90 with a different insertion site on the skin surface and still access the same receiving cup 46. This can prevent the user from accessing the port 40 at the same insertion site over two consecutive access events. This allows the skin at a first insertion location (“a”) to fully heal before accessing the port again at the first site (“a”).

In an embodiment, rotating the dial 130 in a first direction (e.g. clockwise) can cause the first guide cylinder 120A and the second guide cylinder 120B to rotate in the same direction, either in the first direction or in a second direction opposite the first direction. In an embodiment, rotating the dial 130 in a first direction can cause the first guide cylinder 120A and the second guide cylinder 120B to rotate in opposite directions.

In an embodiment, first guide cylinder 120A and the second guide cylinder 120B can be co-aligned. For example, as shown in FIG. 3A, the first needle channel 122A on the first guide cylinder 120A, and the second needle channel 122B disposed on second guide cylinder 120B can both be positioned at the “12 o'clock” position. In an embodiment, rotating the dial 130 can cause the first guide cylinder 120A and the second guide cylinder 120B to rotate concurrently in one of the first direction or the second direction. For example, rotating dial 130 can cause the first needle channel 122A and the second needle channel 122B to both rotate from the “12 o'clock position” to a “1 o'clock position.” As will be appreciated, these positions are exemplary and are provided for ease of explanation, these positions are not intended to be limiting in any way.

In an embodiment, rotating the dial 130 can cause the first guide cylinder 120A and the second guide cylinder 120B to rotate in opposite directions. For example, as shown in FIGS. 3A-3B, rotating the dial 130 can cause the first needle channel 122A to move from a “12 o'clock” position to a “1 o'clock” position, and cause the second needle channel 122B to move from a “12 o'clock” position to an “11 o'clock” position. These and other combinations of dial 130, guide cylinder 120, or needle channel 122 positions or rotational directions, are also contemplated.

In an embodiment, the guide cylinder 120 can rotate about the central axis 80 between the first position and the second position by an arc distance of between 1° and 359°. In an embodiment, the guide cylinder 120 can rotate about the central axis 80 between the first position and the second position by an arc distance of between 1° and 180°. However, greater or lesser arc distances are also contemplated. In an embodiment, the guide cylinder 120 can rotate about the central axis 80 between the first position and the second position by a distance of between 5° and 20°.

In an embodiment, the dial 130 can include graduated markings, ridges or detents to indicate when the guide cylinder 120 is in one of the first position or the second position. In an embodiment, the dial 130 can include a ball and detent system, ratchet system, cam system, or the like, such that the dial 130 can be rotated between one or more predetermined positions. As such, rotating the dial 130 between the one or more predetermined positions can cause the guide cylinders 120A, 120B to be rotated between one or more predetermined positions. In an embodiment, the device 100 can include one or more gear mechanisms, as described herein, configured to allow the dial 130 to be rotated in a first direction but to prevent rotation of the dial 130 in a second direction, opposite the first direction. Advantageously, this can prevent the user from rotating the dial back to a previous, recently used position and can prevent the user from re-accessing an insertion site prematurely.

In an embodiment, the stabilization device 100 can include an adhesive layer disposed on a portion of the bottom surface 116 and configured to adhere to the skin surface 20 and secure the device 100 in position over the port 40. In an embodiment, the device 100 can include an anchor pad 132 coupled to a bottom surface 116 of the device. The anchor pad 132 can include an adhesive layer disposed on bottom surface thereof and configured to adhere to the skin surface 20 and secure the device 100 in position over the port 40. Advantageously, the anchor pad 132 can stabilize the device 100 over the port 40 once it is positioned correctly without requiring the user to hold the device in place.

In an embodiment, the guide cylinder 120 can be triggered to automatically rotate by removal of the needle 90 from the needle channel 122. For example, the device 100 can include the guide cylinder disposed at a first position. The needle 90 can be inserted into the needle channel 122 and access the receiving cup 46 by piercing the skin surface at the first location “a.” Once dialysis has been completed, the user can remove the needle 90 from the needle channel 122. Removing the needle from the channel 122 can trigger the guide cylinder 120 to automatically rotate from the first position to the second position without the user actuating the dial 130. Advantageously, the device 100 can automatically advance to a different position ready for a subsequent access event. This can mitigate the user accidently using the same position for two consecutive access events.

In an embodiment, the device 100 can include a ratchet system to prevent retrograde rotation of the guide cylinder 120 from the second position back to the previous, first position. This can prevent the user from accidentally rotating the dial 130 or the guide cylinder in an opposite direction and using the same position for two consecutive access events, or re-accessing the port at a previous insertion site before the site has had time to fully heal.

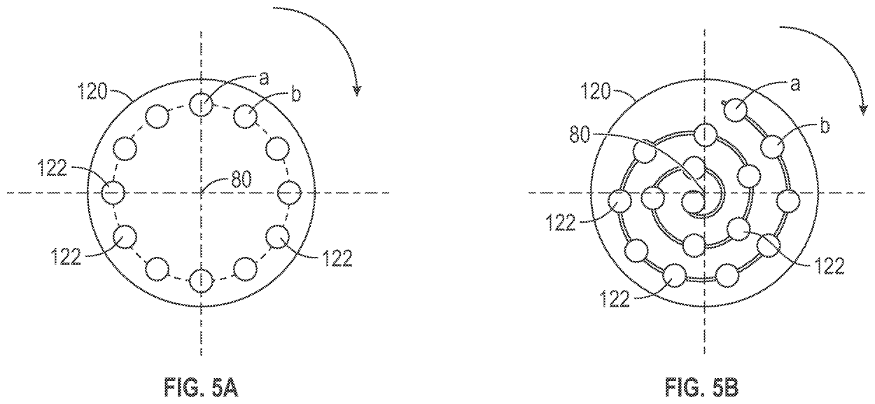

In an embodiment, as shown in FIG. 5A, the needle channel 122 can rotate in a circular path about the central axis 80 such that the needle channel 122 is positioned at one or more different positions about the circular path. To note, the needle channels 122 shown in FIGS. 5A-5B illustrate a single needle channel 122 at varying positions. Advantageously, the plurality of insertion sites disposed about the circular path can allow a first access site (“a”) to fully heal before re-accessing the port at the first site again, mitigating the formation of scar tissue or “buttonholes.”

In an embodiment, the guide cylinder 120 can be rotated about the central axis 80 by an irregular distance such that guide cylinder 120 must rotate through two or more full rotations (i.e. +360°) before the needle channel 122 realigns with the first insertion site (a) again. In an embodiment, the guide cylinder 120 can be rotated about the central axis 80 in an arc distance of one of 7°, 11°, 13°, or 14°, or a similar angle that is not a factor of 360°. In an embodiment, the guide cylinder 120 can be rotated an arc distance that is a prime number. Advantageously, by rotating the guide cylinder by a non-factorable arc distance, the needle channel 122 would have to complete multiple full rotations about the longitudinal axis of the guide cylinder 120 before aligning with a previously used access site. As such, the previously used access site can be allowed additional time to fully heal before being re-accessed, mitigating the formation of scar tissue or “buttonholes.”

In an embodiment, as shown in FIG. 5B, the guide cylinder 120 can include a cam gear mechanism to rotate the needle channel 122 in a spiral path, elliptical path, star shaped, epicyclic path, random path, or similar non-circular path, about the central longitudinal axis 80. As such the needle channel 122 can be positioned at a different position about cross-sectional area of the guide cylinder 120. These and other combinations needle channel positions and position pathways, including circular, spiral, elliptical, polygonal, or random are also contemplated to fall within the scope of the invention.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A stabilization device for a subcutaneous port, comprising:
a body defining a port recess disposed in a bottom surface thereof, and configured to receive a portion of the subcutaneous port therein in a first orientation; and
a guide cylinder including a needle channel aligned with a receiving cup of the port when the port is engaged with the body in the first orientation, the guide cylinder extending from an outer surface of the body to the port recess and rotationally coupled to the body between a first position and a second position, the needle channel aligned with a first insertion site on the skin surface in the first position and a second insertion site on the skin surface in the second position.

2. The stabilization device according to claim 1, wherein a central axis of the needle channel is offset from a central axis of the guide cylinder.

3. The stabilization device according to claim 1, further including a dial, rotationally coupled with the body and configured to rotate the guide cylinder between the first position and the second position.

4. The stabilization device according to claim 1, wherein an inner surface profile of the port recess is configured to mirror an outer profile of the port to align the guide cylinder with the receiving cup of the port in the first orientation and to prevent the port from engaging the port recess in a second orientation.

5. The stabilization device according to claim 1, wherein an arc distance between the first position and the second position is between 1° and 180°.

6. The stabilization device according to claim 1, wherein an arc distance between the first position and the second position is a non-factorable number of degrees of 360°.

7. The stabilization device according to claim 1, wherein an arc distance between the first position and the second position is a prime number.

8. The stabilization device according to claim 1, wherein the guide cylinder is configured to automatically rotate from the first position to the second position when a needle is removed from the needle channel.

9. The stabilization device according to claim 1, further including a ratchet mechanism configured to prevent the guide cylinder from rotating from the second position to the first position.

10. The stabilization device according to claim 1, wherein the needle channel moves in a circular path between the first position and the second position.

11. The stabilization device according to claim 1, wherein the needle channel moves in a non-circular path between the first position and the second position.

12. A method of accessing a subcutaneous port, comprising:
engaging a stabilization device with the subcutaneous port to axially align a guide cylinder of the stabilization device with a receiving cup of the port;
inserting a needle through a needle channel of the guide cylinder to access the receiving cup of the subcutaneous port at a first insertion site;
removing the needle from the stabilization device; and
rotating the guide cylinder of the stabilization device from a first position to a second position to align the needle channel with a second insertion site, different from the first access site.

13. The method according to claim 12, wherein a central axis of the needle channel is offset from a central axis of the guide cylinder.

14. The method according to claim 12, further including rotating a dial from a first position to a second position to rotate the guide cylinder from the first position to the second position.

15. The method according to claim 12, wherein an inner surface profile of a port recess of the stabilization device is configured to mirror an outer profile of the subcutaneous port to align the guide cylinder with the receiving cup of the subcutaneous port in a first orientation and to prevent the port recess from engaging the port in a second orientation, different from the first orientation.

16. The method according to claim 12, wherein an arc distance between the first position and the second position of the guide cylinder is between 1° and 180°.

17. The method according to claim 12, wherein an arc distance between the first position and the second position of the guide cylinder is a non-factorable number of degrees of 360°.

18. The method according to claim 12, wherein an arc distance between the first position and the second position of the guide cylinder is a prime number.

19. The method according to claim 12, wherein removing the needle from the needle channel causes the guide cylinder to automatically rotate from the first position to the second position.

20. The method according to claim 12, wherein the stabilization device includes a ratchet mechanism configured to prevent the guide cylinder from rotating from the second position to the first position.

21. The method according to claim 12, further including rotating the needle channel in a circular path between the first position and the second position.

22. The method according to claim 12, further including rotating the needle channel in a non-circular path between the first position and the second position.
